(19) United States
(12) Patent Application Publication
(10) Pub. No.: US 2004/0143182 A1
(43) Pub. Date: Jul. 22, 2004

(54) System and method for monitoring and stimulating gastro-intestinal motility

(76) Inventors: Pavel Kucera, Lausanne (CH); Vincent Schlageter, Lausanne (CH)

Correspondence Address: NIXON & VANDERHYE, PC, 1100 N GLEBE ROAD, 8TH FLOOR, ARLINGTON, VA, 22201-4714, US

(21) Appl. No.: 10/635,463

(22) Filed: Aug. 7, 2003

Related U.S. Application Data
(60) Provisional application No. 60/402,018, filed on Aug. 8, 2002.
(60) Provisional application No. 60/402,033, filed on Aug. 8, 2002.

(51) Int. Cl. A61B 5/05

(52) U.S. Cl. 600/424

(57) ABSTRACT

A system and method for monitoring and stimulating GI motility is provided. One or more capsules (or motility markers) may be ingested by a patient for passage through the GI tract. Each capsule may contain an emitting coil which produces an AC magnetic field, or a permanent magnet. An external sensing device comprising multiple magnetic field sensors is used to measure, among other data, the position of the ingested capsules within the GI tract. As signals from the magnetic field sensors are acquired, an iterative algorithm continuously calculates the magnetic momentum and position of each capsule in real time. The position of each capsule may be defined by five coordinates (x, y, z, θ, φ) representing three translations and two rotations. This data may be displayed in real time or saved for further processing. When one or more capsules reach a segment of the GI tract that has been identified for treatment, the capsule(s) may be subjected to an external magnetic field applied by a generator or other device. The applied magnetic field may result in movements of the capsule with respect to the enteric nervous system so as to trigger the natural, physiological propulsive reflexes of the GI tract.

### CROSS-REFERENCE TO RELATED APPLICATIONS

This Application claims priority to U.S. Provisional Patent Application Serial No. 60/402,018 filed Aug. 8, 2002, and U.S. Provisional Patent Application Serial No. 60/402,033 filed Aug. 8, 2002, each of which are incorporated herein by reference.

### FIELD OF THE INVENTION

This invention relates to a system and method for monitoring and stimulating motility within the gastrointestinal (“GI”) tract.

### BACKGROUND OF THE INVENTION

With reference to FIG. 1, the GI tract of a human begins with the mouth, where food is ingested, and continues to the esophagus, stomach, small intestine, large intestine, rectum, and anus. The liver, pancreas, and gall bladder (not illustrated) are other organs associated with the GI tract.

Contraction of the longitudinal and circular muscle fibers of the organs comprising the GI tract (i.e., peristalsis) results in the movement of food through the GI tract. The introduction of enzymes and digestive juices at various stages along the GI tract enables food to be broken down, and allows nutrients to be absorbed. In the large intestine, excess fluids (e.g., water) are reabsorbed and stool is formed for eventual excretion.

When peristalsis becomes impaired, normal GI motility may give way to one or more GI motility disorders. Examples of various GI motility disorders include irritable bowel syndrome, constipation, diarrhea, achalasia, chronic intestinal pseudo-obstruction, gastroparesis, and gastroesophageal reflux disease.

Properly diagnosing and treating GI motility disorders, in general, can be quite difficult. Some GI motility disorders, for example, are functional. A functional disorder is a disorder that does not show any evidence of an organic or physical disease, and thus will likely not be detected via blood tests, X-rays, or other diagnostic techniques. Rather, functional disorders may be nervous disorders or disorders which are biochemical in nature, and are often diagnosed based on symptoms.

Other GI motility disorders can be difficult to treat, particularly when the etiology and pathogenesis of the disorder are not elucidated (e.g., chronic constipation).

Several techniques have been developed to assist in the monitoring and analysis of GI motility. Orogastric manometry, for example, generally provides information about the muscular function of the esophagus and stomach, while anal manometry typically only yields information about the muscular function of the descending colon and rectum. Neither, however, is particularly successful in providing information about the muscular function of the small intestine, or the ascending and transverse colon of the large intestine. The invasive nature of manometry may also make it a less desirable option for patients.

GI motility is also analyzed using radiology and/or other imaging techniques. For instance, some methods track ingested non-dissolving markers through the GI tract via X-ray. Unfortunately, this method does not allow for the continuous monitoring of GI motility due to the dangers associated with prolonged exposure to X-rays.

Biomagnetic techniques have also been implemented to study GI motility. Such techniques, however, typically rely on expensive and sensitive equipment [e.g., a Superconducting Quantum Interference Device (SQUID)] to measure the magnetic fields created when the muscles of the GI tract contract and produce electrical currents. These and other drawbacks exist with known GI motility monitoring techniques.

For many GI motility disorders, medical treatment often seeks to restore the normal peristaltic movements of the GI tract through laxatives, prokinetics, and changes in diet. These methods, however, are often unpredictable, unreliable, or ineffective.

Recent techniques for stimulating GI motility focus on electrical stimulation. Patients afflicted with gastroparesis, for instance, may have gastric pacemakers implanted. Although promising, this technique is still quite invasive.

As another example, electrical stimulation of the colon via electrodes has been attempted in animal experiments to alleviate constipation. One drawback associated with this technique, however, is that the surgical implantation of electrodes is an invasive procedure that may be subject to complications including, among other things, infection. Additionally, the placement of electrodes cannot be changed without re-operation. These and other drawbacks exist with known GI motility stimulation techniques.

Accordingly, it would be advantageous to provide a system and method for monitoring and stimulating GI motility that is minimally invasive, and that overcomes at least the aforementioned drawbacks of known techniques.

### SUMMARY OF THE INVENTION

The invention solving these and other problems relates to a system and method for monitoring and stimulating GI motility.

According to an embodiment of the invention, to monitor GI motility, one or more capsules (or motility markers) may be ingested by a patient for passage through the GI tract. The capsules may be ingested at one time, or at pre-determined time intervals such that they remain spaced apart within the GI tract.

In one embodiment, each capsule may comprise or contain an emitting coil which produces an AC magnetic field. Each ingested capsule may emit a signal at a different frequency (e.g., frequency multiplexing) or at a different time (e.g., time multiplexing) than the others so as to uniquely identify (via sensors) each of the capsules as they pass through the GI tract. Alternatively, each capsule may comprise or contain a permanent magnet (e.g., rare earth cylindrical magnet) as the source for the magnetic field.

One advantage of using emitting coils as markers is that they mitigate the inhomogenity of the earth's magnetic field and serve to reduce external magnetic perturbations. The use of permanent magnets as markers, however, may also be advantageous as the use of magnets eliminates the need for either a power supply within the capsules or for a source of external excitation. Additionally, capsules having magnets rather than emitting coils may be smaller, thus facilitating clinical applications with children and/or small animals.

According to an embodiment of the invention, an external sensing device comprising multiple magnetic field sensors (e.g., an array of inductive sensors) is used to measure, among other data, the position of the ingested capsules within the GI tract via their magnetic fields. In a clinical setting, the sensing device may be mounted on an adjustable support structure capable of positioning the sensing device in alignment with one or more segments of the GI tract. Alternatively, the sensing device may be incorporated in a belt that may be worn by a patient in both clinical and non-clinical (e.g., at home) settings.

As signals from the magnetic field sensors are acquired, an iterative algorithm continuously calculates the magnetic momentum and position of each capsule in real time. The position of each capsule may be defined by five coordinates (x, y, z, θ, φ) representing three translations and two rotations. This data may be displayed in real time or saved for further processing. In an embodiment wherein an emitting coil is placed within each capsule, the addition of a second emitting coil positioned orthogonal to the first allows for recovery of a sixth degree of freedom (i.e., the rotation around the symmetry axis of the first emitting coil).

According to an embodiment of the invention, to stimulate GI motility, the progression of one or more capsules through the GI tract may first be monitored using a monitoring system similar to that described above. Other GI motility monitoring techniques may be used. Upon reaching a segment of the GI tract that has been identified for treatment, a capsule may be subjected to an external magnetic field applied by a generator or other device. The applied magnetic field may result in movements of the capsule with respect to the digestive mucosa (enteric nervous system) so as to trigger the natural, physiological propulsive reflexes of the GI tract.

In one embodiment, both a generator and a sensing device may be aligned with one or more segments of the GI tract. The generator and sensing device may comprise one integral unit, or two separate units. Alternatively, the generator and sensing device may be incorporated in a belt that can be worn by a patient. Other configurations may of course be implemented.

According to an embodiment of the invention, the generator may apply an external magnetic field at a user-selected, controlled frequency and intensity. The generator may be operated directly by a physician, researcher, patient or other individual. Alternatively, a processor may be programmed to calculate the position of a capsule within the GI tract in real-time, and transmit activation/de-activation signals (wired or wireless) to the generator when the capsule has reached a targeted treatment site. Accordingly, GI motility may be stimulated at any location within the GI tract in an effective, minimally invasive manner in both clinical and non-clinical settings.

One advantage provided by the invention is the use of autonomous ingested capsules to monitor the motility of the GI tract. Ingested capsules travel through the GI tract along with content (e.g., ingested food) so as to provide a more accurate measure of GI motility. In addition, if one or more of the capsules contains a drug, the drug may be released at a given location along the GI tract.

Another advantage of the invention is that multiple capsules, when ingested at pre-determined time intervals, can provide valuable information regarding the reflex and coordination between different segments of the GI tract. For example, at any given time, a patient may have one capsule in the stomach, one in the small intestine, and one in the colon. In this regard, information concerning the activities of the stomach, small intestine, and colon at any one point in time may be analyzed.

Yet another advantage of the invention is the ability to define the position of each capsule using five coordinates (x, y, z, θ, φ). This enables physicians and/or researchers to gather and analyze valuable information. For instance, displacement of each capsule (and thus displacement of the content of the GI tract) may be studied along with small movements of the walls of the organs of the GI tract which tend to result in rotations of a capsule.

Still yet another advantage of the invention is the ability to easily and effectively monitor motility of the GI tract in both clinical and non-clinical settings.

An additional advantage provided by the invention is that GI motility may be stimulated using a system and method that is minimally invasive with little risk of complications.

Another advantage provided by the invention is that GI motility may be stimulated at any location within the GI tract at any time.

Still yet another advantage of the invention is the ability to easily and effectively stimulate motility of the GI tract in both clinical and non-clinical settings.

These and other objects, features, and advantages of the invention will be apparent through the detailed description of the preferred embodiments and the drawings attached hereto. It is also to be understood that both the foregoing general description and the following detailed description are exemplary and not restrictive of the scope of the invention.

### BRIEF DESCRIPTION OF THE DRAWINGS

FIG. 1 is an exemplary illustration of the GI tract of a human.

FIG. 2 illustrates one or more capsules (or motility markers), according to an embodiment of the invention.

FIGS. 3A-3B are exemplary illustrations of various implementations of a GI motility monitoring system, according to an embodiment of the invention.

FIG. 4 depicts a grid illustrating at least five coordinates (x, y, z, θ, φ) that may be used to define the position of a capsule (or motility marker), according to an embodiment of the invention.

FIG. 5 illustrates a system for monitoring and stimulating GI motility, according to an embodiment of the invention.

### DETAILED DESCRIPTION OF PREFERRED EMBODIMENTS

The following description sets forth various embodiments of a system and method for monitoring and stimulating GI motility. Although these embodiments are described with reference to the GI tract of a human, it should be understood that one or more aspects of the invention described herein may be modified or adapted for use with various animals.

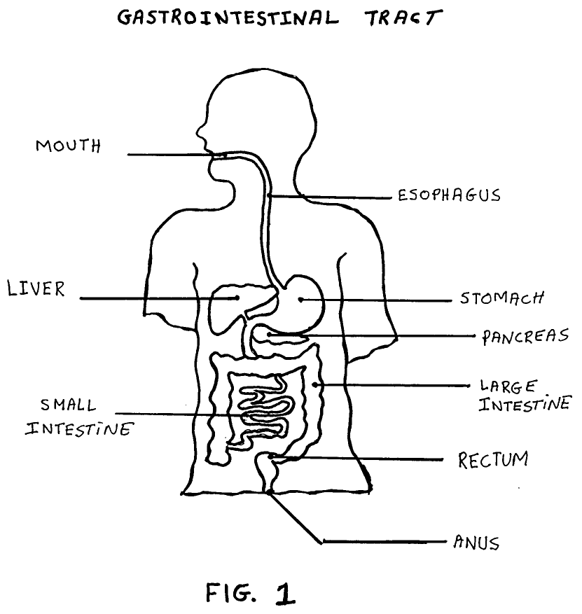

As illustrated in FIG. 2, one or more capsules (10a, 10b, . . . 10n) may be ingested by a patient. Each capsule may be ingested approximately simultaneously, or at pre-determined time intervals to allow them to be spaced apart within the GI tract. Each capsule may include a biocompatible coating 14 comprising any known material that facilitates ingestion, and is suitable for passage along the GI tract. It should be understood that the term “capsule” may be used interchangeably herein with “marker,” as the position of the one or more capsules (10a, 10b, . . . 10n), when ingested, may be marked or traced as the capsules travel through the GI tract with other content (e.g., ingested food).

According to an embodiment of the invention, each of the one or more capsules (10a, 10b, . . . 10n) may comprise or contain an emitting coil (e.g., 10b) that produces an AC magnetic field. The magnetic field produced is approximately equal to a magnetic field produced by an ideal magnetic dipole. In various embodiments, the emitting coils of the one or more capsules (10a, 10b, . . . 10n) may be configured so as to emit a signal at a different frequency (e.g., frequency multiplexing) or at a different time (e.g., time multiplexing) from the others. This enables magnetic field sensors (described below) to uniquely identify each of the one or more capsules (10a, 10b, . . . 10n) as they pass through the GI tract.

The use of emitting coils as markers is advantageous as they mitigate the inhomogenity of the earth's magnetic field and serve to greatly reduce external noise. In other words, little or no calibration is needed to account for the magnetic field of the earth. Batteries may be used as energy sources for the emitting coils. Other energy sources may also be used. Alternatively, the emitting coils may comprise pickup coils which obtain energy from a source external to the body. Thus, an internal battery or capacitor can be recharged. Other configurations are possible.

According to one embodiment, the one or more capsules (10a, 10b, . . . 10n) may comprise or contain permanent magnets such as, for example, rare earth cylindrical magnets. The use of magnets eliminates the need for a power supply or external excitation. Additionally, capsules having magnets rather than emitting coils may be smaller, thus facilitating clinical applications with children and/or small animals.

In yet another embodiment, each of the one or more capsules (10a, 10b, . . . 10n) may be attached to a catheter or retractable string.

Generally, the use of autonomous ingested capsules is advantageous in that they travel through the GI tract along with content (e.g., ingested food) so as to provide a more accurate measure of GI motility. Further, the use of multiple capsules, which may be ingested simultaneously or at spaced intervals, can provide valuable information regarding the reflex and coordination between different segments of the GI tract. For example, at any given time, a patient may have a capsule (e.g., capsule “10a”) in the stomach, one in the small intestine (e.g., “lob”), and one in the colon (e.g., “10n”). Thus, information about what occurs in the small intestine while the stomach and colon are engaged in particular functions, for example, may be analyzed.

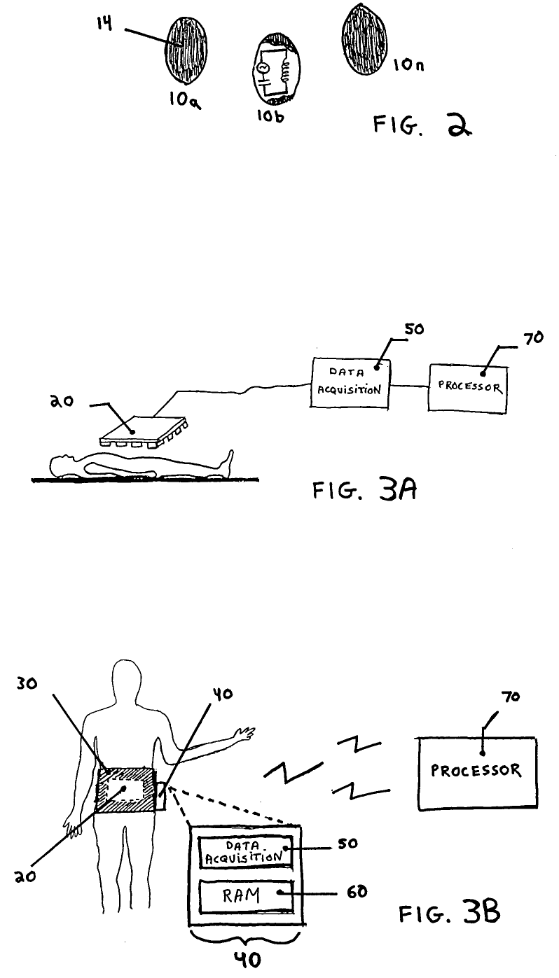

FIGS. 3A-3B are exemplary illustrations of various implementations of the GI motility monitoring system. As shown in FIG. 3A, a patient may ingest one or more capsules (10a, 10b, . . . 10n) in a clinical setting. The patient may then be oriented in either a horizontal position (as depicted) or a vertical position with respect to an external sensing device 20. Sensing device 20 may be mounted on an adjustable support structure (not illustrated) that facilitates alignment with one or more segments of the GI tract.

According to an embodiment of the invention, sensing device 20 may comprise an array of inductive sensors whose position with respect to one another is fixed. As one example, sensing device 20 may comprise sixteen Hall sensors arranged in a 4×4 array. Other sensors (e.g., magneto-resistive or flux-gate) or sensor configurations may be used.

As the one or more capsules (10a, 10b, . . . 10n) progress through the GI tract, AC magnetic field signals emitted by the coils therein are measured by sensing device 20. As the frequency and phase of the transmitted waves may fluctuate, sensing device 20 may re-create these characteristics by combining signals from several or all of the sensors. The signals from each sensor comprising sensing device 20 may be sampled at a predetermined frequency (e.g., 10 Hz. or greater) and filtered or amplified as necessary by data acquisition electronics 50. Data acquisition electronics 50 may further convert the filtered and/or amplified analog signals to digital signals, and transmit them to a processor 70 via a communication link (wired or wireless) for further processing. Additionally, data acquisition electronics 50 may include a multiplexing circuit for multiplexing signals emitted from the coils at different frequencies from one another (frequency multiplexing) or at different times (time multiplexing). If frequency multiplexing is used, the emitting coils within each capsule may be configured to emit signals continuously, or configured to cycle on and off to conserve energy.

A supplementary coil may be attached to the patient's thorax (xyphoid) to serve as an external landmark to position the sensor matrix and also record the patient's ventilation. Respiratory artifacts may also be corrected using an accelerometer or a nostril thermistance.

The configuration of the components illustrated in FIG. 3A, and described in detail above, may vary according to different embodiments. For example, in one embodiment, sensing device 20 and data acquisition electronics 50 may be contained within a first structure or housing that is coupled to processor 70 via a wired or wireless communication link. Alternatively, data acquisition electronics 50 and processor 70 may be housed together within, for example, a computing device that is coupled to sensing device 20 via a wired or wireless communication link. Other configurations may exist.

According to an embodiment of the invention illustrated in FIG. 3B, sensing device 20 may be incorporated in a belt 30 that may be worn by a patient. A patient may ingest one or more capsules (10a, 10b, . . . 10n) and don belt 30 such that it is positioned around, for example, the abdomen. The patient may then either lie down, ambulate, or engage in a combination of both.

According to one embodiment, a mobile data pack 40 may be integral with (or detachably coupled to) belt 30. Mobile data pack 40 may include data acquisition electronics 50 (as described above) as well as a Random Access Memory (RAM) 60. If the GI motility of the patient is being monitored in a clinical setting, data acquisition electronics 50 may, as described above, send data to processor 70 in real-time via a wired or wireless communication link. By contrast, if a patient is away from a clinical setting for a predetermined period of time (e.g., at home for 24 hours), acquired data may be stored in RAM 60 for subsequent download to processor 70. Other configurations and implementations are possible.

Processor 70 may include any one or more of, for instance, a personal computer, portable computer, PDA (personal digital assistant), or other processing device. As signals from sensing device 20 are received, processor 70 may execute an iterative algorithm (e.g., the Levenberg-Marquardt optimization algorithm) that continuously calculates the position of each of the one or more capsules (10a, 10b, . . . 10n) as they travel through the GI tract. This data may be generated in real-time during a clinical session, or generated after data acquired and stored in RAM 60 has been subsequently downloaded to processor 70.

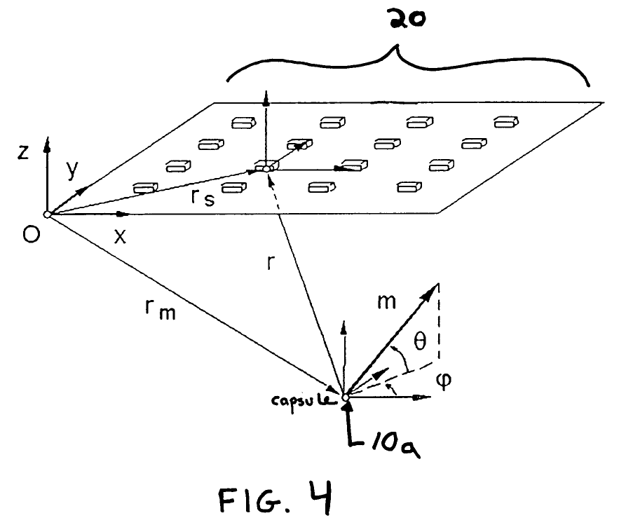

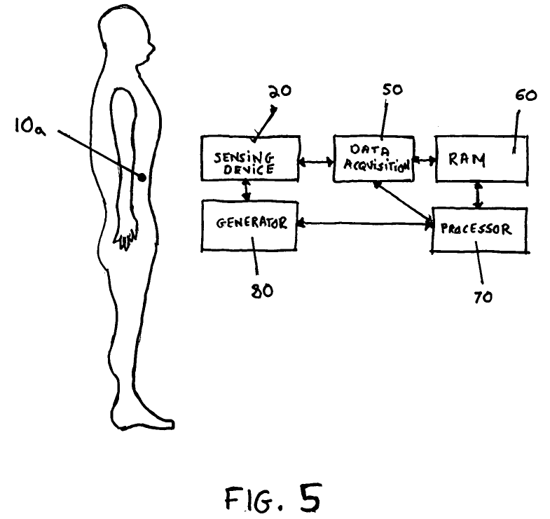

According to an embodiment of the invention illustrated in FIG. 4, the position of each capsule (e.g., capsule “10a” as illustrated) may be defined by five coordinates (x, y, z, θ, φ) representing three translations and two rotations. This information may be displayed in two dimensions versus time (2D v. t) or in three dimensions (3D) in real-time via a monitor or other display device associated with processor 70. Other display parameters may be used. This information may also be saved for further processing.

The ability to acquire positional data defined by five coordinates for each capsule enables physicians and/or researchers to gather and analyze valuable information. For instance, displacement of each capsule (and thus displacement of the content of the GI tract) may be studied along with small movements of the walls of the organs of the GI tract which tend to result in rotations of a capsule.

According to an embodiment of the invention, the addition of a second emitting coil positioned orthogonal to the first in the one or more capsules (10a, 10b, . . . 10n) allows for recovery of a sixth degree of freedom (i.e., the rotation around the symmetry axis of the first emitting coil).

In alternative embodiments, the one or more capsules (10a, 10b, . . . 10n) may be configured to measure additional parameters including, for example, temperature, pressure, and pH. This information may be transmitted outside of the body using the same emitting coil, and by modulating the frequency, amplitude, or phase of the emitted signals. Other embodiments may exist.

Medical treatment for many GI motility disorders often focuses on restoring the normal peristaltic movements of the GI tract. As illustrated in FIG. 5, a system for monitoring and stimulating GI motility is provided. This system is similar to the systems shown in FIGS. 3A-3B (and described above), yet further comprises an external magnetic field generator 80.

Generally, the progression of one or more capsules (e.g., capsule “10a”) through the GI tract is monitored using any of the embodiments described in detail above. For the purpose of stimulation, capsule 10a may preferably include or comprise a small permanent magnet. Other GI motility monitoring techniques may be used. Upon reaching a segment of the GI tract that has been identified for treatment, capsule 10a is subjected to an external magnetic field applied by generator 80. The applied magnetic field may result in movements of capsule 10a with respect to the digestive mucosa (enteric nervous system). In particular, the gastrointestinal mechanoreceptors may be stimulated to trigger the natural, physiological propulsive reflexes of the GI tract.

According to an embodiment, a patient may ingest one or more capsules (10a, 10b, . . . 10n). Alternatively, each of the one or more capsules (10a, 10b, . . . 10n) may be attached to a catheter or retractable string. Once the capsules are within the GI tract, the patient may then be oriented in either a vertical position (as shown), or in a horizontal position with respect to external sensing device 20 and generator 80.

The configuration of the components illustrated in FIG. 5 may vary according to different embodiments. For example, in one embodiment, both generator 80 and sensing device 20 may comprise one integral unit mounted on an adjustable support structure (not illustrated) that may be aligned with one or more segments of the GI tract. Alternatively, sensing device 20 and generator 80 may be separate and thus mounted on individual, adjustable support structures (not illustrated) that may each be aligned with one or more segments of the GI tract. In addition, both sensing device 20 and generator 80 may be incorporated in a belt (similar to belt 30 depicted in FIG. 3B) that can be worn by a patient. As yet another alternative, only generator 80 may be incorporated in a belt while sensing device 20 is positioned via a separate adjustable support structure, or vice versa. Other configurations may of course be implemented.

Similarly, data acquisition electronics 50, ram 60, and processor 70 may be interconnected via wired or wireless communication links in a number of configurations, including those described above with reference to FIGS. 3A-3B.

According to an embodiment of the invention, generator 80 may apply an external magnetic field at a user-selected, controlled frequency and intensity. Generator may 80 be operated directly by a physician, researcher, patient or other individual. Alternatively, processor 70 may be programmed to calculate the position of a capsule (e.g., capsule “10a”) within the GI tract in real-time, and transmit activation/de-activation signals (wired or wireless) to generator 80 when capsule 10a has reached a targeted treatment site. Accordingly, GI motility may be stimulated at any location within the GI tract in an effective, minimally invasive manner in both clinical and non-clinical settings.

Other embodiments, uses and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. A system for monitoring motility of a patient's gastrointestinal tract, comprising:
at least one capsule sized to be ingested by a patient, the at least one capsule adapted to generate a magnetic field;
a sensing device, positioned external to the patient's body, for measuring the magnetic field of the at least one capsule as the at least one capsule progresses through the patient's gastrointestinal tract; and
a processor, operatively connected to the sensing device, for receiving signals from the sensing device and calculating the at least one capsule's magnetic momentum and position within the gastrointestinal tract.

2. The system of claim 1, wherein the at least one capsule houses an emitting coil that produces a high frequency magnetic field.

3. The system of claim 2, wherein the at least one capsule comprises two capsules, and wherein the emitting coil of each capsule emits a signal at a frequency different from that of the other.

4. The system of claim 2, wherein the at least one capsule comprises two capsules, and wherein the emitting coil of each capsule emits a signal at a time different from that of the other.

5. The system of claim 1, wherein the at least one capsule houses a permanent magnet that produces a magnetic field.

6. The system of claim 1, wherein the at least one capsule is coated with a biocompatible coating.

7. The system of claim 1, wherein the sensing device comprises an array of inductive sensors.

8. The system of claim 7, wherein the sensing device comprises a 4×4 array of sensors, and wherein the sensors comprise either Hall sensors, magneto-resistive sensors, or flux-gate sensors.

9. The system of claim 1, wherein the sensing device is incorporated into a belt that is worn by the patient.

10. The system of claim 9, wherein the belt is positioned around the patient's abdomen.

11. The system of claim 1, wherein the processor receives signals from the sensing device in real-time.

12. The system of claim 1, wherein signals from the sensing device are stored in a random access memory during a session, and then downloaded to the processor subsequent to the termination of the session.

13. The system of claim 1, wherein the processor executes an iterative algorithm that continuously calculates the magnetic momentum and position of the at least one capsule as it progresses through the gastrointestinal tract.

14. The system of claim 13, wherein the position of the at least one capsule is defined by five coordinates (x, y, z, θ, φ) representing three translations and two rotations.

15. The system of claim 1, wherein the at least one capsule houses a first emitting coil and a second emitting coil positioned orthogonal to the first emitting coil, and wherein both the first and second emitting coils produce a high frequency magnetic field.

16. The system of claim 15, wherein the processor executes an iterative algorithm that continuously calculates the magnetic momentum and position of the at least one capsule as it progresses through the gastrointestinal tract, and wherein the position of the at least one capsule is defined by six coordinates representing three translations and three rotations.

17. The system of claim 1, further comprising a magnetic field generator, positioned external to the patient's body, adapted to generate a magnetic field when the at least one capsule has reached a targeted treatment site within the patient's gastrointestinal tract.

18. The system of claim 18, wherein the magnetic field generated by the magnetic field generator results in movements of the at least one capsule with respect to digestive mucosa of the patient's gastrointestinal tract so as to stimulate gastrointestinal motility.

19. A method for monitoring motility of a patient's gastrointestinal tract, the method comprising the steps of:
providing at least one capsule to a patient for ingestion, the at least one capsule adapted to generate a magnetic field;
positioning a sensing device external to the patient's body for measuring the magnetic field of the at least one capsule as the at least one capsule progresses through the patient's gastrointestinal tract; and
transmitting signals from the sensing device to a processor to enable the processor to calculate the at least one capsule's magnetic momentum and position within the gastrointestinal tract.

20. The method of claim 19, wherein the at least one capsule houses an emitting coil that produces a high frequency magnetic field.

21. The method of claim 20, wherein the at least one capsule comprises two capsules, and wherein the emitting coil of each capsule emits a signal at a frequency different from that of the other.

22. The method of claim 20, wherein the at least one capsule comprises two capsules, and wherein the emitting coil of each capsule emits a signal at a time different from that of the other.

23. The method of claim 19, wherein the at least one capsule houses a permanent magnet that produces a magnetic field.

24. The method of claim 19, wherein the at least one capsule is coated with a biocompatible coating.

25. The method of claim 19, wherein the sensing device comprises an array of inductive sensors.

26. The method of claim 25, wherein the sensing device comprises a 4×4 array of sensors, and wherein the sensors comprise either Hall sensors, magneto-resistive sensors, or flux-gate sensors.

27. The method of claim 19, wherein the sensing device is incorporated into a belt that is worn by the patient.

28. The method of claim 27, further comprising the step of positioning the belt around the patient's abdomen.

29. The method of claim 19, wherein the step of transmitting signals from the sensing device to the processor occurs in real-time.

30. The method of claim 19, wherein the step of transmitting signals from the sensing device to the processor further comprises the steps of:
storing signals from the sensing device in a random access memory during a session; and
downloading the signals to the processor subsequent to the termination of the session.

31. The method of claim 19, wherein the processor executes an iterative algorithm that continuously calculates the magnetic momentum and position of the at least one capsule as it progresses through the gastrointestinal tract.

32. The method of claim 31, wherein the position of the at least one capsule is defined by five coordinates (x, y, z, θ, φ) representing three translations and two rotations.

33. The method of claim 19, wherein the at least one capsule houses a first emitting coil and a second emitting coil positioned orthogonal to the first emitting coil, and wherein both the first and second emitting coils produce a high frequency magnetic field.

34. The method of claim 33, wherein the processor executes an iterative algorithm that continuously calculates the magnetic momentum and position of the at least one capsule as it progresses through the gastrointestinal tract, and wherein the position of the at least one capsule is defined by six coordinates representing three translations and three rotations.

35. The method of claim 19, further comprising the step of:
positioning a magnetic field generator external to the patient's body, the magnetic field generator adapted to generate a magnetic field when the at least one capsule has reached a targeted treatment site within the patient's gastrointestinal tract.

36. The method of claim 35, wherein the magnetic field generated by the magnetic field generator results in movements of the at least one capsule with respect to digestive mucosa of the patient's gastrointestinal tract so as to stimulate gastrointestinal motility.

37. A system for monitoring motility of a patient's gastrointestinal tract, comprising:
at least two capsules to be ingested by a patient at pre-determined time intervals, the at least two capsules each housing an emitting coil adapted to generate a high frequency magnetic field;
a sensing device, positioned external to the patient's body, for measuring the magnetic field of the at least two capsules as the at least two capsules progress through the patient's gastrointestinal tract; and
a processor, operatively connected to the sensing device, for receiving signals from the sensing device and executing an iterative algorithm that continuously calculates the magnetic momentum and position of the at least two capsules in real-time as they progress through the patient's gastrointestinal tract.

38. The system of claim 37, wherein the emitting coil of each of the at least two capsules emits a signal at a frequency different from that of the other.

39. The system of claim 37, wherein the emitting coil of each of the at least two capsules emits a signal at a time different from that of the other.

40. The system of claim 37, wherein the position of each of the at least two capsules is defined by five coordinates (x, y, z, θ, φ) representing three translations and two rotations.

41. A system for stimulating motility of a patient's gastrointestinal tract, comprising:
at least one capsule sized to be ingested by a patient, the at least one capsule housing a permanent magnet that produces a magnetic field;
means for monitoring the progress of the at least one capsule through the patient's gastrointestinal tract; and
means for applying an external magnetic field when the at least one capsule has reached a targeted treatment site within the patient's gastrointestinal tract such that the external magnetic field results in movements of the at least one capsule with respect to digestive mucosa of the patient's gastrointestinal tract to stimulate gastrointestinal motility.
